(19) United States
(12) Patent Application Publication
(10) Pub. No.: US 2011/0020881 A1
(43) Pub. Date: Jan. 27, 2011

(54) METHOD OF PRODUCING BIOFUEL USING BROWN ALGAE

(75) Inventors: Hwa Young CHO, Hwaseong-si (KR); Byung Jo YU, Hwaseong-si (KR); Jae Chan PARK, Yongin-si (KR); Sung Min PARK, Yongin-si (KR); Jae Hwa LEE, Busan (KR)

Correspondence Address: CANTOR COLBURN LLP, 20 Church Street, 22nd Floor, Hartford, CT, 06103, US

(73) Assignee: SAMSUNG ELECTRONICS CO., LTD, Suwon-si (KR)

(21) Appl. No.: 12/704,148

(22) Filed: Feb. 11, 2010

(30) Foreign Application Priority Data
Jul. 24, 2009 (KR) 10-2009-0067863

(51) Int. Cl. C12P 19/02; C12N 9/14

(52) U.S. Cl. 435/105; 435/195

(57) ABSTRACT

In a method of producing biofuel using brown algae, Bacterium antarctica is used as a hydrolysis catalyst for saccharification to obtain monosaccharides from the brown algae. The saccharification with the hydrolysis catalyst is effective in saccharification of the brown algae.

### CROSS-REFERENCE TO RELATED APPLICATION

This application claims priority to Korean Patent Application No. 10-2009-0067863, filed on Jul. 24, 2009, and all the benefits accruing therefrom under 35 U.S.C. §119, the contents of which in its entirety is hereby incorporated by reference.

### BACKGROUND

1. Field

The disclosures herein relate to a method of producing biofuel using brown algae and technology based on an isolated polypeptide capable of degrading brown algae.

2. Description of the Related Art

With globally increasing concern about the exhaustion of resources and pollution of the environment by overuse of fossil fuels, the development of new and renewable alternative energy resources that stably and continuously produce energy are being considered. In the ongoing development of such alternative energy resources, the technology for producing biofuel from biomass has been attracting considerable attention.

Today, biomass capable of producing biofuel is derived from grains such as sugar cane and corn, and from wood sources, which are by-products in forestry and agriculture. However, wood biomass has a limit in production due to the matters of environmental disruption such as food competition and devastation of soil, limited cultivation area and difficulty in supplying nutrients.

On the other hand, production of biofuel using algae, which are abundant ocean resources compared to land resources, has economical and environmental advantages. Algae have a significantly higher growth rate and productivity per unit area than land plants. Thus, new and renewable energy resources such as ethanol and butanol, hydrogen and methane may be produced using brown algae, waste algae may be effectively treated, and surplus resources of algae are effectively available.

In 2008, the total production of algae in Korea was 934,890 tons in both shallow-sea cultures and distant waters fisheries. Domestically, algae are produced in a culture area of about 76,183 hectares (ha). Thus, in view of the fact that Korea's exclusive economic zone is 44,900,000 ha, it may be possible to achieve an increase in the production of algae. The production of algae will further increase due to construction of seaweed beds and development of the cultivation technology of algae.

### SUMMARY

In one aspect, a method of producing biofuel is provided. The method includes saccharifying the brown algae with at least one hydrolysis catalyst to produce monosaccharides. The at least one hydrolysis catalyst, is selected from the following: a) Bacterium antarctica; b) a culture solution of Bacterium antarctica; c) a supernatant prepared by centrifuging the culture solution of Bacterium antarctica; and d) a lysate of Bacterium antarctica cells.

The hydrolysis catalysts include Bacterium Antarctica, which is a microorganism capable of hydrolyzing brown algae.

The method may also include pre-treating the brown algae to extract polysaccharides before saccharification and/or fermenting the monosaccharides obtained by saccharification to produce biofuel such as bioethanol.

In one embodiment, the method of producing biofuel includes pre-treating the brown algae to produce polysaccharides, wherein the pretreating comprises treating brown algae biomass with heat and/or acid; adding at least one hydrolysis catalyst to the polysaccharides, wherein the at least one hydrolysis catalyst is selected from Bacterium antarctica; a culture solution of Bacterium antarctica; a supernatant prepared by centrifuging the culture solution of Bacterium antarctica; and a lysate of Bacterium antarctica cells; saccharifying the polysaccharides to produce monosaccharides; and fermenting the monosaccharides using microorganisms.

In another aspect, an isolated polypeptide from a Bacterium species is disclosed. The isolated polypeptide has hydrolyzing activity of brown algae, and is isolated from pure-cultures of Bacterium species that generate or activate alginase. The Bacterium species includes a 16S rRNA having at least about 95% sequence identity to a sequence corresponding to Bacterium antarctica strain AL-1 deposited with the Genebank of the Korea Research Institute of Bioscience and Biotechnology under accession number KCTC 11531 BP.

The isolated polypeptide is capable of generating or activating the enzyme alginase for degrading alginate abundant in brown algae biomass, thereby hydrolyzing the brown algae. Thus, brown algae may be degraded using the isolated polypeptide.

### BRIEF DESCRIPTION OF THE DRAWINGS

The above and other aspects, advantages and features of this disclosure will become more apparent by describing in further detail exemplary embodiments thereof with reference to the accompanying drawings, in which. It should be understood that various aspects of the drawings may have been exaggerated for clarity:

FIG. 1 is a graph showing the cell growth of Bacterium antarctica strain AL-1 over time as described in Experimental example 1 (X axis: Time(h), and Y axis: O.D 600 nm);

FIG. 2 is a graph showing the reducing sugar concentration produced in various species of brown algae by hydrolysis using Bacterium antarctica strain AL-1 as described in Experimental example 2 (X axis: Time(h), and Y axis: Reducing sugar concentration(g/L));

FIG. 3 is a graph showing the reducing sugar concentration produced in Laminaria japonica, Sargassum felvellnm and Hizikia fusiformis using supernatants (Ex. 1) or cell lysates (Ex. 3) from Bacterium antarctica strain AL-1, as described in Experimental example 2 (Y axis: Reducing sugar concentration (g/L));

FIG. 4 is a graph showing bioethanol production from Laminaria japonica using S. cerevisiae (Comp. Ex.: comparative example, Ex: example) (X axis: Time(h), and Y axis: Ethanol production(g/L));

FIG. 5 is a graph showing bioethanol production from Laminaria japonica using P. tannophilus (Comp. Ex.: comparative example, Ex: example) (X axis: Time(h), and Y axis: Ethanol concentration(g/L));

FIG. 6 is a graph showing bioethanol production from Sargassum felvellnm using S. cerevisiae (Comp. Ex.: comparative example, Ex: example) (X axis: Time(h), and Y axis: Ethanol concentration(g/L));

FIG. 7 is a graph showing bioethanol production from Sargassum felvellnm using P. tannophilus (Comp. Ex.: comparative example, Ex: example) (X axis: Time(h), and Y axis: Ethanol concentration(g/L)); and

FIG. 8 is a graph showing bioethanol production from Hizikia fusiformis using P. tannophilus (Comp. Ex.: comparative example, Ex: example) (X axis: Time(h), and Y axis: Ethanol concentration(g/L)).

### DETAILED DESCRIPTION

Various exemplary embodiments will now be described more fully with reference to the accompanying drawings in which exemplary embodiments are shown This invention may however, be embodied in many different forms, and should not be construed as limited to the embodiments set forth herein. Rather, these embodiments are provided so that this disclosure will be thorough and complete, and will fully convey the scope of the invention to those skilled in the art.

It will be understood that when an element is referred to as being “on” “connected to” another element or layer, the element or layer can be directly on or connected to another element or layer or intervening elements or layers. In contrast, when an element is referred to as being “directly on” another element, there are no intervening elements present. As used herein, the term “and/or” includes any and all combinations of one or more of the associate listed items.

It will be understood that, although the terms first, second, third etc. may be used herein to describe various elements, components, regions, layers and/or sections, these elements, components, regions, layers or sections should not be limited by these terms. These terms are only used to distinguish one element from another element, component, region, layer or section. Thus, a first element, component, region, layer or section could be termed a second element, component, region, layer or section without departing from the teachings of the present invention.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e. meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

In one aspect, a method for biologically hydrolyzing brown algae using Bacterium antarctica is disclosed.

To produce biofuel using brown algae, polysaccharides existing in brown algae first must be hydrolyzed and converted into monosaccharides through the process of saccharification.

Examples of the brown algae may include, but are not limited to, Laminaria japonica, Sargassum fulvellum, Hizikia fusiformis, Ecklonia cava, Undaria pinnatifida, Analipus japonicus, Chordaria flagelliformis, Ishige okamurae, Scytosiphon lomentaria, Endarachne binghamiae, Ecklonia stolonifera, Eisenia bicyclis, Costaria costata Saunders, Sargassum horneri, and Sargassum thunbergii.

The brown algae contain polysaccharides such as alginate and laminaran, and monosaccharides such as mannitol. The polysaccharide content in the brown algae varies according to season, species, and cultivation environment. For example, it is known that the content of alginate in brown algae is generally the highest in January to March, and the content of laminaran and mannitol are the highest in August to October.

Alginate is a high viscosity element and a high molecular weight polysaccharide composed of β-1,4-D-mannuronic acids linked by 1,4-glycosidic bonds. Laminaran is a storage polysaccharide of brown algae, and composed of glucan having β-1,3 bonds. Laminaran may be hydrolyzed to produce glucose. Thus, biofuel can be produced by fermenting this degradation product.

In one embodiment, a method of producing biofuel includes saccharifying the brown algae with at least one hydrolysis catalyst to produce monosaccharides. The at least one hydrolysis catalyst is selected from the group consisting of:

Bacterium antarctica,

a culture solution of Bacterium antarctica,

a supernatant prepared by centrifuging the culture solution of Bacterium antarctica and

a lysate of Bacterium antarctica cells.

The catalyst for hydrolysis includes or uses a Bacterium antarctica.

The Bacterium antarctica strain described herein comprises a 16S rRNA having at least about 95% sequence identity to a sequence corresponding to Bacterium antarctica strain AL-1 which was deposited with the Genebank of the Korea Research Institute of Bioscience and Biotechnology under accession number KCTC 11531 BP.

In one embodiment, the base sequence of the 16S rRNA may include (i) a base sequence having at least about 95% sequence identity to the base sequence of SEQ ID NO: 1, or (ii) a base sequence having at least about 95% sequence identity to a base sequence hybridized with the base sequence of SEQ ID NO: 1 under stringent conditions.

The base sequence of SEQ ID NO: 1 is a DNA sequence corresponding to the 16S rRNA of Bacterium antarctica strain AL-1 deposited with the Genebank of the Korea Research Institute of Bioscience and Biotechnology under accession number KCTC 11531 BP.

In another embodiment, the Bacterium antarctica described herein is Bacterium antarctica strain AL-1 (hereinafter, in some cases, referred to as “strain AL-1”) deposited with the Genebank of the Korea Research Institute of Bioscience and Biotechnology under accession number KCTC 11531 BP.

Bacterium antarctica may be pure-cultured from seawater and brown algae. In one exemplary embodiment, Bacterium antarctica may be cultured by spreading a sample containing seawater and brown algae on a multi-layer plate medium, and then isolating the Bacterium antarctica from the plate.

The multi-layer plate medium may include, for example, a lower layer composed of 2.5% (w/v) of NaCl, 0.1% (w/v) of KH₂PO₄, 0.05% (w/v) of FeSO₄.7H₂O, 0.05% (w/v) of KCl, 0.1% (w/v) of NH₄Cl, and 2% (w/v) of agar, and an upper layer composed of 1% (w/v) of sodium alginate and 2% (w/v) of agar.

The culture solution of Bacterium antarctica may be obtained by inoculating and growing Bacterium antarctica cells in a liquid medium containing alginate, laminaran, and peptone.

The culturing conditions are not particularly limited, and the culture, for example, of Bacterium antarctica may be performed at a temperature of about 20 to about 35° C. for a period of about 12 to about 60 hours.

The supernatant of Bacterium antarctica may be obtained by centrifuging the culture solution of Bacterium antarctica. In an exemplary embodiment, the supernatant may be obtained by centrifuging the culture solution at a speed of about 10,000 to about 15,000 rotations per minute (rpm) for a period of about 1 to about 30 minutes.

The lysate of Bacterium antarctica may be obtained by disintegrating the Bacterium antarctica cells in the culture solution using a known disintegrator. For example, the lysate may be obtained by ultrasonic disintegration using a sonicator or by repeating several cycles of disintegration-pause.

The hydrolysis catalyst may be used alone, or in combination with various known degradative enzymes according to the type and composition of the biomass. Examples of degradative enzymes include, but are not limited to, β-agarase, β-galactosidase, β-glucosidase, endo-1,4-β-glucanase, α-amylase, β-amylase, glucoamylase and cellulase.

The saccharification process may be performed at a temperature of about 60 to about 200° C. for a period of about 0.5 to about 8 hours.

In one embodiment, the method of producing biofuel may include pretreating the brown algae for obtaining polysaccharides before the saccharification. In another embodiment, the method may further include fermenting the monosaccharides obtained after the saccharification to produce biofuel.

In one embodiment, the method of producing biofuel may include pre-treating the brown algae biomass with heat and/or acid to obtain polysaccharides; saccharifying the polysaccharides to produce monosaccharides by adding at least one hydrolysis catalyst to the polysaccharides; and fermenting the monosaccharides using microorganisms to produce biofuel. The at least one hydrolysis catalyst is selected from the group consisting of Bacterium Antarctica, a culture solution of Bacterium Antarctica, a supernatant prepared by centrifuging the culture solution of Bacterium Antarctica, and a lysate of Bacterium antarctica cells.

The method of pre-treating for extracting polysaccharides from the brown algae is not performed by a particular method, and thus may be performed by a method known in the art.

In one exemplary embodiment, for the pretreatment, the brown algae may be immersed in an acidic solvent. The reaction temperature may be from about 25 to about 150° C. Examples of the acidic solvents include, but are not limited to, sulfuric acid, chloric acid, hydrobromic acid, nitric acid, acetic acid, formic acid, perchloric acid, phosphoric Acid, p-toluenesulfonic acid (PTSA) and commercial solid acid.

Alternatively, the brown algae may be immersed in a basic solvent for the pretreatment. Examples of the basic solvents include, but are not limited to, potassium hydroxide, sodium hydroxide, calcium hydroxide and an aqueous ammonium solution.

In another exemplary embodiment, for the pretreatment, the brown algae may be heated at a high temperature.

A brown algae substrate, for example, may be dipped into distilled water, and then heated in an autoclave at a temperature of about 100 to about 200° C. for a period of about 10 to about 80 minutes. In some cases, the heated brown algae substrate may be milled using a known pulverizer to make the substrate accessible by the hydrolysis catalyst.

In yet another embodiment, the brown algae substrate may be washed to remove any contaminants, and then dried, and milled into powder to be applied to the pretreatment and/or saccharification. The brown algae substrate may be dried using a hot air dryer or dried by air dry.

In one embodiment, the saccharification may use an additional hydrolysis catalyst, other than the hydrolysis catalyst as described above.

For example, an acidic catalyst may be added during the saccharification using the hydrolysis catalyst as described above.

Examples of the acidic catalysts include, but are not limited to, sulfuric acid, chloric acid, hydrobromic acid, nitric acid, acetic acid, formic acid, perchloric acid, phosphoric acid, p-toluenesulfonic acid (PTSA) and commercial solid acid. The acidic catalyst may be added to the reaction at a concentration of about 0.05 to about 50 weight % (wt %), and saccharified at a temperature of about 80 to about 300° C.

The hydrolysis catalyst and the acidic catalyst are not added in a particular order. Thus, the hydrolysis catalyst and the acidic catalyst may be added in a multi-step process, or simultaneously added. For example, an acidic catalyst may be added first, and then the hydrolysis catalyst may be added. Alternatively, the hydrolysis catalyst may be added first and then the acidic catalyst may be added.

In one embodiment, fermentation is performed to ferment the monosaccharides using microorganisms such as yeast or bacteria, and thereby convert the monosaccharides into biofuel.

The microorganism may be, but is not limited to, yeast selected from the group consisting of the genera of Saccharomyces, Pachysolen, Clavispora, Kluyveromyces, Debaryomyces, Schwannniomyces, Candida, Pichia and Dekkera.

In one embodiment, the fermentation may be performed using the yeast strains Saccharomyces cerevisiae, Pachysolen tannophilus, Sarcina ventriculi, Kluyveromyces fragilis, Zygomomonas mobilis, Kluyveromyces marxianus IMB3, or Brettanomyces custersii, which are effective in ethanol fermentation.

In an exemplary embodiment, the fermentation may be performed using the yeast strains Saccharomyces cerevisiae (S. cerevisiae) or Pachysolen tannophilus (P. tannophilus).

Alternatively, the fermentation may be performed using the bacterial strains Clostridium acetobutylicum, Clostridium beijerinckii, Clostriduim aurantibutylicum, or Clostridium tetanomorphum, which are effective in butanol or acetone fermentation.

The biofuel produced may be, for example, alcohols having 1 to 4 carbon atoms or ketones having 2 to 4 carbon atoms. The alcohol, for example, may be methanol, ethanol, propanol or butanol, and the ketone may be acetone.

In another exemplary embodiment, an isolated polypeptide, isolated from a pure-culture of Bacterium species that generates or activates alginase, is capable of hydrolyzing brown algae, the Bacterium species includes a 16S rRNA having at least about 95% sequence identity to a sequence corresponding to Bacterium antarctica strain AL-1 deposited with the Genebank of the Korea Research Institute of Bioscience and Biotechnology under accession number KCTC 11531 BP.

The isolated polypeptide is capable of generating or activating the enzyme alginase, degrading alginate abundant in brown algae biomass, and thereby hydrolyzing brown algae. Accordingly, the polysaccharides in brown algae may be degraded into monosaccharides using the isolated polypeptide.

The term “polypeptide” refers to a peptide or protein containing two or more amino acids linked to each other by peptide bonds or by modified peptide bonds. The “polypeptide” includes short chains such as peptides, oligopeptides or oligomers, and to long chains such as proteins. The “polypeptide” may include amino acids other than the 20 gene-encoded amino acids. The “polypeptide” includes amino acid sequences modified by natural processes or by chemical modification techniques known in the art. The modifications to the “polypeptide” include acetylation, acylation, ADP-ribosylation, amidation, biotinylation, covalent attachment of flavin, covalent attachment of a heme moiety, covalent attachment of a nucleotide or nucleotide derivative, covalent attachment of a lipid or lipid derivative, covalent attachment of phosphotidylinositol, crosslinking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

The term “isolated” when used to describe polypeptides, refers to a polypeptide that has been identified and separated and/or recovered from a component of its natural environment. For example, a protein naturally existing in the original living organism is not “isolated,” but the same type of protein removed from the natural coexisting substance is “isolated.” The term also embraces recombinant polypeptides and chemically synthesized polypeptides. Further, a polynucleotide encoding for a polypeptide, a polypeptide or a protein introduced into a living organism by transformation, genetic manipulation or by other recombination techniques is considered “isolated” even though it is present in a living organism.

The Bacterium species may be Bacterium antarctica strain AL-1 deposited under accession number KCTC 11531 BP in the Genebank of the Korea Research Institute of Bioscience and biotechnology (KRIBB; Yuseong-gu, Daejeon, Korea) on 21 Jul., 2009.

The 16S rRNA comprises (i) a base sequence having at least about 95% sequence identity to the base sequence of SEQ ID NO: 1, or (ii) a base sequence having at least about 95% sequence identity to a base sequence hybridized with the base sequence of SEQ ID NO: 1 under stringent conditions.

The term “stringent conditions” refers to conditions given when a gene is incubated for a period of about 2.5 hours in a solution containing 6× standard sodium citrate (SSC) and 0.1% Sodium dodecyl sulfate (SDS) at a temperature of 42° C., and then a filter is washed in 1.0×SSC/0.1% SDS at a temperature of 65° C.

The term “identity” reflects the relationship between two or more polypeptide or polynucleotide sequences, and is determined by comparing the sequences to one another. Generally, the term “identity” refers to an exact nucleotide to nucleotide or amino acid to amino acid correspondence between the two or more polynucleotide sequences or the two or more polypeptide sequences, respectively, over the length of the compared sequences. Methods of comparing identity and similarity of two sequences are known in the art.

For example, the percent (%) identity between two polynucleotides, and the % identity and % similarity between two polypeptide sequences may be determined using the Wisconsin Sequence Analysis Package, version 9.1 such as the programs BESTFIT and GAP (Devereux J. et al., Nucleic Acids Res, 12, 387-395 (1984); available from Genetics Computer Group, Madison Wis., USA).

Other programs for determining identity and/or similarity between sequences include the BLAST family of programs (Altschul S. F. et al., J. Mol. Biol., 215, 403-410 (1990); Altschul S. F. et al., Nucleic Acids Res., 25:389-3402 (1997); available from National Center for Biotechnology Information (NCBI), Bethesta, Md., USA and accessible through the website of NCBI at www.ncbi.nlm nih gov) and FASTA (Pearson W. R., Methods in Enzymology, 183, 63-99 (1990); Pearson W. R. and Lipman D. J., Proc Nat. Acad. Sci. USA, 85, 2444-2448, 19988, available as partial Wisconsin Sequence Analysis Package).

### EXAMPLES

### Preparation Example 1

### Heat Pretreatment of Brown Algae

2 g each of powder of Laminaria japonica, Sargassum fulvellum or Hizikia fusiformis is added to 100 ml of distilled water, and heated in an autoclave for 15 minutes at 121° C. for high pressure sterilization.

### Preparation Example 2

### Acid Pretreatment of Brown Algae

2 g each of powder of Laminaria japonica, Undaria pinnatifida, Sargassum fulvellum, Ecklonia cava, Hizikia fusiformis or Pachymeniopsis elliptica is added to 0.1 N HCl (80 ml), and heated in an autoclave at 121° C. for 30 minutes for high pressure sterilization. Afterwards, each sample is stirred at 150 rpm for 1 hour at 30° C., and neutralized with sodium hydroxide to adjust the pH to 6.5 to 7.

### Preparation Example 3

### Isolation of Bacterium antarctica AL-1 Strain

As a sample for isolating a strain capable of hydrolyzing brown algae, seawater and brown algae obtained from Kijang, Busan, South Korea are used. An aliquot (100 μl) of the sample solution diluted 100 times (e.g. 1/100 dilution) is spread on a multi-layer plate medium, and cultured in an incubator at a constant temperature of 30° C. for 48 h, followed by isolating the AL-1 strain from a single colony having a large clean zone.

The multi-layer plate medium, having an upper and a lower layer is used as the isolation medium. The lower layer is prepared using 25 g/L of NaCl, 1.0 g/L of KH₂PO₄, 0.5 g/L of FeSO₄.7H₂O, 0.5 g/L of KCl, 1.0 g/L of NH₄Cl and 20 g/L of agar, adjusted to have a pH of 7.0. The upper layer is prepared using 10 g/L of sodium alginate and 20 g/L of agar, adjusted to have a pH of 7.0.

### Preparation Example 4

### Preparation of Culture Solution of AL-1 Strain

A colony of Bacterium antarctica strain AL-1 is inoculated into a 300-ml Erlenmeyer flask containing 100 ml of a medium containing 8 g/L of sodium alginate, 8 g/L of laminaran and 5 g/L of peptone, and then shaken to culture, which is performed at 27° C. for 48 hours.

### Preparation Example 5

### Preparation of Supernatant of AL-1 Strain

The culture solution prepared as described in Preparation example 4 is centrifuged. The solution is centrifuged at 12,000 rpm for 10 minutes using a Hanil Science industrial Micro 17TR. Following centrifugation only the supernatant (excluding the cell mass) is removed.

### Preparation Example 6

### Preparation of Lysate of AL-1 Strain

The culture solution prepared as described in Preparation example 4 is disintegrated using a Sonic Dismembrator Model 500 (Fisher Scientific Co.). Disintegration (e.g. sonication) is conducted at an ultrasonic wave intensity of 70% in three cycles of 15-sec disintegration and 30-sec pause.

### Experimental Example 1

### Change in Enzyme Activity According to Culture Period of AL-1 Strain

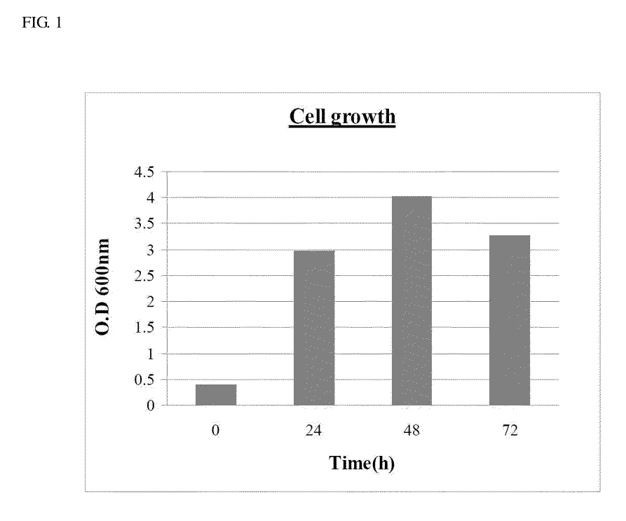

To identify changes in enzyme activity during the culture period of the AL-1 strain, AL-1 cells are cultured in a liquid medium containing 8 g/L of alginate, 8 g/L of laminaran, and 5 g/L of peptone as described in Preparation example 4. Cell growth is measured by taking a sample from the culture solution at intervals of 24 hours, isolating and washing the cells and measuring the optical density. The optical density (OD) is measured using an UV spectrometer (A₆₀₀ nm), and the results are shown in FIG. 1.

Referring to FIG. 1, it can be seen that cell growth of the AL-1 strain is most activated at 48 hours after initiation of the culture.

### Example 1

Saccharification is performed by adding 5 ml of the supernatant prepared as described in Preparation example 5 to each of the samples of Laminaria japonica, Sargassum fulvellum and Hizikia fusiformis prepared as described in Preparation example 1.

### Example 2

Saccharification is performed by adding 5 ml of the culture lysates prepared as described in Preparation example 6 to each of the samples of Laminaria japonica, Sargassum fulvellum and Hizikia fusiformis prepared as described in Preparation example 1.

### Comparative Example 1

Saccharification is performed by adding 2 g each of the powder of Laminaria japonica, Sargassum fulvellum or Hizikia fusiformis to 100 ml of distilled water and heating the reaction mixture in an autoclave at 121° C. for 15 minutes.

### Comparative Example 2

Saccharification is performed by adding 2 g of the powders of Laminaria japonica, Sargassum fulvellum or Hizikia fusiformis to 80 ml of 0.1 N HCl, heating the reaction mixture in an autoclave at 121° C. for 30 minutes for high pressure sterilization, stirring the reaction mixture at 30° C. at 150 rpm for 1 hour, and neutralizing the mixture using sodium hydroxide to adjust the pH to 6.5 to 7.

### Experimental Example 2

### Measurement of Degradation Capability of Alginate of AL-1 Strain and Reducing Sugar Concentration

A supernatant prepared as described in Preparation example 5 and six substrates of Laminaria japonica, Undaria pinnatifida, Sargassum fulvellum, Ecklonia cava, Hizikia fusiformis and Pachymeniopsis elliptica are used. The supernatant (500 μl) and 1.0 ml of each brown algae substrate, are mixed with each other for a 30-minute reaction at 30° C. Reducing sugar (monosaccharide) concentration is measured by a dinitrosalicylic acid (DNS) assay method.

The dinitrosalicylic acid assay method is performed by adding 2 ml of a DNS solution to 500 μl of a sample, heating the mixture at 95° C. for 10 minutes and then measuring optical density at a wavelength of 540 nm. Standard calibration curves for both alginate and laminaran are plotted by quantifying reducing sugars generated using maltose and glucose, respectively. One unit of enzyme is determined as an amount of enzyme producing 1 μmol of reducing sugars per 1 minute.

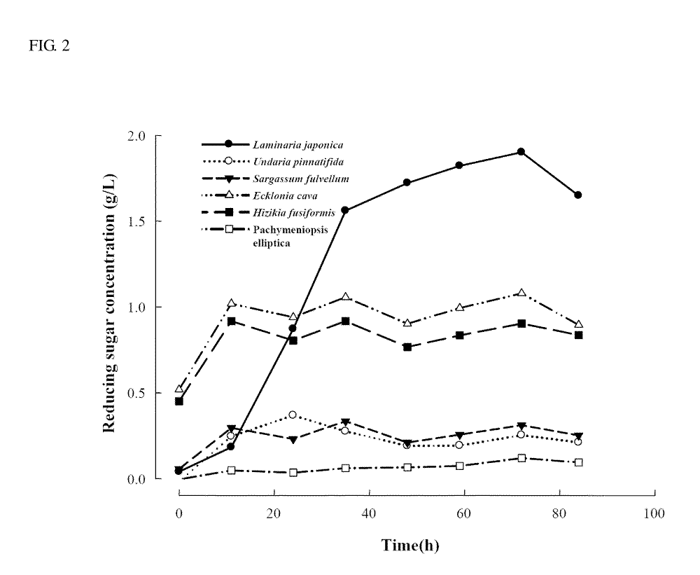

Hydrolysis according to the passage of time is measured by quantifying the amount of reducing sugars produced over a period of 84 hours, which is shown in FIG. 2.

Referring to FIG. 2, in hydrolysis of brown algae using the AL-1 strain, the highest hydrolysis activity is exhibited in Laminaria japonica. Hydrolysis of Undaria pinnatifida, Sargassum fulvellum, Ecklonia cava, Hizikia fusiformis, and Pachymeniopsis elliptica is saturated at 12 hours, but hydrolysis of Laminaria japonica is steadily increased for up to 72 hours. As a result, reducing sugar is obtained at a concentration of 1.900 g/L (concentration obtained after 72 hours of the hydrolysis) from the sample of Laminaria japonica.

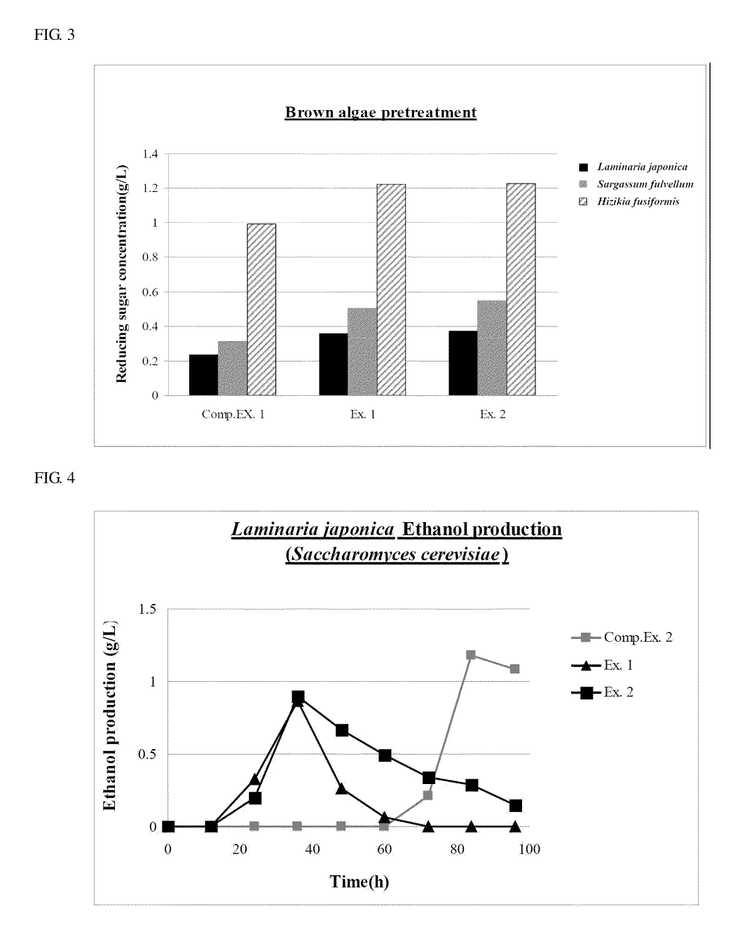

In addition, after saccharification is performed according to Examples 1 and 2 and Comparative example 1 with respect to Laminaria japonica, Sargassum fulvellum and Hizikia fusiformis, the total production of reducing sugar is measured, and the result is shown in FIG. 3.

Referring to FIG. 3, it can be seen that production of reducing sugar increases when a supernatant or cell lysate of AL-1 strains is used, as compared when AL-1 strains are only thermally treated as described in Comparative example 1. Among the samples of brown algae, Hizikia fusiformis shows high reducing sugar production. There is no difference in enzymatic hydrolysis between Examples 1 (supernatant) and 2 (cell lysate).

### Experimental Example 3

### Measurement of Bioethanol Production in Laminaria japonica

Saccharification in Laminaria japonica is performed according to Examples 1 and 2 and Comparative example 2. Specifically, 3 ml of S. cerevisiae is inoculated into each saccharified solution. A volume of 100 ml of distilled water is added for fermentation by mixing at a speed of 150 rpm and a temperature of 30° C. A sample is taken at time intervals of 12 hours from the fermented solution to measure bioalcohol production over time. The results are shown in FIG. 4.

To quantify the produced bioethanol, a fermented sample is centrifuged at 12,000 rpm for 10 minutes, and then a supernatant is analyzed using gas chromatography (GC). The GC is performed using an HP 5890 series II and HP-FFAP (cross-linked PEG-TPA, specification: 30 m/0.25 mm/0.25 μl) as a column. For the GC, N₂ is used as the mobile phase at a flow rate of 0.6 ml/min, the injection temperature is 100° C., and the detector temperature is 200° C. The increasing temperature conditions are: 50° C. (1.4 min); (10° C./min); 60° C. (1 min); (25° C./min); 100° C. (1 min); (50° C./min); and 150° C. (1 min).

Referring to FIG. 4, the maximum level of ethanol is produced at about 70 hours after the initiation of saccharification as performed according to Comparative example 2, and thus the processing time becomes longer. However, when the saccharification is performed as described according to the Examples, initial production of ethanol is very high, from which it can be seen that a relatively high production rate and high process efficiency are achieved.

Further, after the saccharification of Laminaria japonica performed according to Examples 1 and 2 and Comparative examples 2, a volume of 3 ml of P. tannophilus is inoculated into each saccharified solution, and then 100 ml of distilled water is added for fermentation by mixing at a speed of 150 rpm and a temperature of 30° C. At a time intervals of 12 hours, bioethanol production is measured, and the results are shown in FIG. 5.

Referring to FIG. 5, when the saccharification is performed as described according to Examples, bioethanol production was significantly higher than Comparative example 1 in which only heat treatment is performed, and initial bioethanol production is also very high.

### Experimental Example 4

### Measurement of Bioethanol Production in Sargassum fulvellum

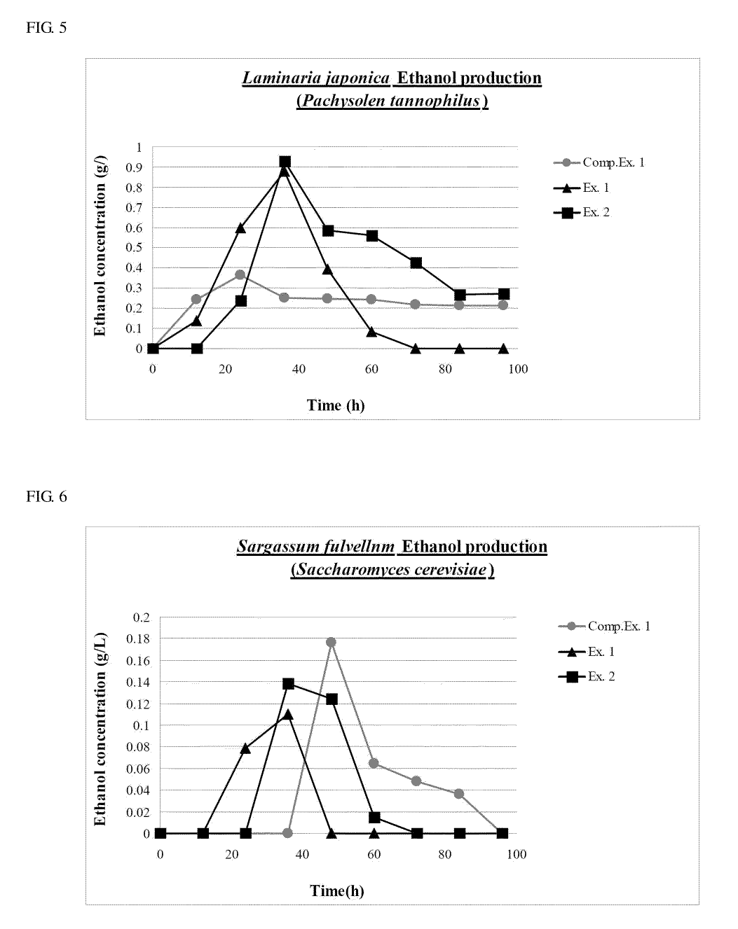

After the saccharification is performed on Sargassum fulvellum as described according to Examples 1 and 2 and Comparative example 1, a volume of 3 ml of S. cerevisiae is inoculated into each saccharified solution, and 100 ml of distilled water is added for fermentation by mixing at a speed of 150 rpm and a temperature of 30° C. At time intervals of 12 hours, bioethanol production according to time is measured, and the result is shown in FIG. 6.

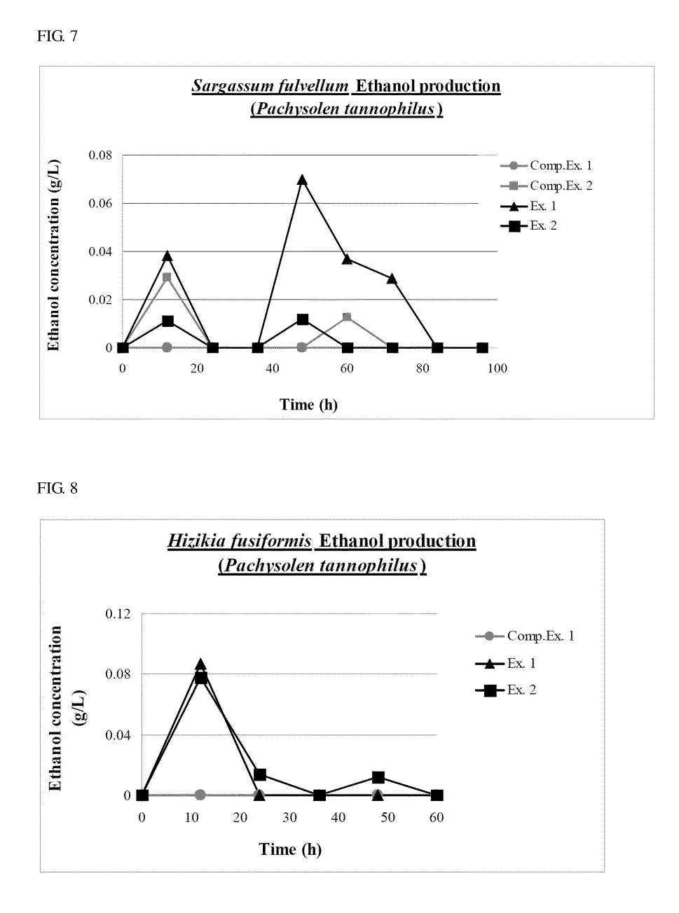

In addition, after the saccharification is performed on Sargassum fulvellum according to Examples 1 and 2 and Comparative examples 1 and 2, a volume of 3 ml of P. tannophilus is inoculated into each saccharified solution, and 100 ml of distilled water is added for fermentation by mixing at a speed of 150 rpm and a temperature of 30° C. At time intervals of 12 hours, bioethanol production according to time is measured, and the result is shown in FIG. 7.

Referring to FIGS. 6 and 7, when the saccharification is performed using either S. cerevisiae or P. tannophilus as fermentable microorganisms according to Examples, ethanol production is higher than Comparative examples 1 and 2 in both cases, and initial ethanol production is also very high.

### Experimental Example 5

### Measurement of Bioethanol Production in Hizikia fusiformis

After the saccharification is performed on Hizikia fusiformis as described according to Examples 1 and 2 and Comparative example 1 and 2, a volume of 3 ml of P. tannophilus is inoculated into each saccharified solution, and 100 ml of distilled water is added for fermentation by mixing at a speed of 150 rpm and a temperature of 30° C. At time intervals of 12 hours, bioethanol production according to time is measured, and the results are shown in FIG. 8.

Referring to FIG. 8, in Comparative example 1, no bioethanol is produced, but in the Examples, bioethanol is effectively produced using Hizikia fusiformis.

Biofuel may be successfully produced on an industrial scale by effectively saccharifying brown algae, which are abundant ocean resources, by enzymatic degradation when using an isolated polypeptide capable of hydrolyzing brown algae described herein and a method of producing bioalcohol using the same.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variation as appropriate, and the inventors intend for the invention to be practice otherwise than as specifically described herein. Accordingly, this invention includes all modification and equivalents of the subject matter recited in the claims appended hereto as permitted by applicable law. Moreover, any combination of the above-described elements in all possible variations thereof is encompassed by the invention unless otherwise indicated herein or otherwise clearly contradicted by law.

While exemplary embodiments have been disclosed herein, it should be understood that other variations may be possible. Such variations are not to be regarded as a departure from the spirit and scope of exemplary embodiments of the present application, and all such modifications as would be obvious to one skilled in the art are intended to be included within the scope of the following claims.

What is claimed is:

1. A method of producing biofuel using brown algae, comprising
saccharifying the brown algae with at least one hydrolysis catalyst to produce monosaccharides;
wherein the at least one hydrolysis catalyst is selected from the group consisting of:
Bacterium antarctica;
a culture solution of Bacterium antarctica;
a supernatant prepared by centrifuging the culture solution of Bacterium antarctica; and
a lysate of Bacterium antarctica cells.

2. The method according to claim 1, wherein the brown algae is at least one strain selected from the group consisting of Laminaria japonica, Sargassum fulvellum, Hizikia fusiformis, Ecklonia cava, Pachymeniopsis elliptica, Ecklonia stolonifera, Eisenia bicyclis, Sargassum thunbergii, and Undaria pinnatifida.

3. The method according to claim 1, wherein the Bacterium antarctica comprises a 16S rRNA having at least about 95% sequence identity to a sequence corresponding to Bacterium antarctica strain AL-1 deposited with the Genebank of the Korea Research Institute of Bioscience and Biotechnology under Accession number KCTC 11531 BP.

4. The method according to claim 3, wherein a base sequence of the 16S rRNA is set forth in SEQ ID NO: 1.

5. The method according to claim 1, wherein the Bacterium antarctica is Bacterium antarctica strain AL-1 deposited with the Genebank of the Korea Research Institute of Bioscience and Biotechnology under Accession number KCTC 11531 BP.

6. The method according to claim 1, wherein the Bacterium antarctica is cultured by
spreading a sample comprising seawater and brown algae on a multi-layer plate medium, and
isolating the Bacterium antarctica,
wherein the multi-layer plate medium comprises a lower layer comprising 2.5% (w/v) of NaCl, 0.1% (w/v) of KH₂PO₄, 0.05% (w/v) of FeSO₄.7H₂O, 0.05% (w/v) of KCl, 0.1% (w/v) of NH₄Cl, and 2% (w/v) of agar and an upper layer comprising 1% (w/v) of sodium alginate and 2% (w/v) of agar.

7. The method according to claim 1, wherein the culture solution of the Bacterium antarctica is prepared by inoculating the Bacterium Antarctica into a medium comprising alginate, laminaran and peptone and culturing the Bacterium antarctica.

8. The method according to claim 7, wherein the culturing is performed at a temperature of about 20 to about 35° C. for about 12 to about 60 hours.

9. The method according to claim 1, wherein the supernatant is obtained by centrifuging the culture solution of the Bacterium antarctica at about 10,000 to about 15,000 rpm for about 1 to about 30 minutes.

10. The method according to claim 1, wherein the lysate of the Bacterium antarctica cells is prepared by disintegrating the Bacterium antarctica cells present in the culture solution using a sonicator.

11. The method according to claim 1, wherein an acidic catalyst is further added during the saccharifying.

12. The method according to claim 1, further comprising pre-treating the brown algae before the saccharification to obtain polysaccharides.

13. The method according to claim 12, wherein the pre-treating comprises heating brown algae biomass at a high temperature or treating brown algae biomass with acid.

14. The method according to claim 1, further comprising fermenting the monosaccharides using a microorganism.

15. The method according to claim 14, wherein the microorganism is Saccharomyces cerevisiae, Pachysolen tannophilus, or a combination thereof.

16. An isolated polypeptide having hydrolyzing activity of brown algae, wherein said polypeptide is isolated from a pure-culture of Bacterium species that generate or activate alginase, the Bacterium species comprising a 16S rRNA having at least about 95% sequence identity to a sequence corresponding to Bacterium antarctica strain AL-1 deposited with the Genebank of the Korea Research Institute of Bioscience and Biotechnology under accession number KCTC 11531 BP.

17. The isolated polypeptide according to claim 16, wherein the Bacterium species is the Bacterium antarctica strain AL-1 deposited with the Genebank of the Korea Research Institute of Bioscience and Biotechnology under accession number KCTC 11531 BP.

18. The isolated polypeptide according to claim 16, wherein the 16S rRNA comprises a base sequence having at least about 95% sequence identity to a base sequence of SEQ ID NO: 1 or a base sequence having at least about 95% sequence identity to a base sequence hybridizing with the base sequence of SEQ ID NO: 1 under stringent conditions.


SEQUENCE LISTING (1 sequence)

SEQ ID NO: 1 - DNA, 1022 bp, bacterium Antarctica AL-1
  rRNA (1)..(1022): 16S ribosomal RNA gene
ggctcagattgaacgctggcggcaggcctaacacatgcaagtcgagcggaaacgaagagtagcttgctactctggcgtcgagcggcggacgggtgagtaatgcttgggaacatgccttgaggtgggggacaacagttggaaacgactgctaataccgcataatgtctacggaccaaagggggcttcggctctcgcctttagattggcccaagtgggattagctagttggtgaggtaatggctcaccaaggcgacgatccctagctggtttgagaggatgatcagccacactgggactgagacacggcccagactcctacgggaggcagcagtggggaatattgcacaatgggcgaaagcctgatgcagccatgccgcgtgtgtgaagaaggccttcgggttgtaaagcactttcagtcaggaggaaaggttagtagttaatacctgctagctgtgacgttactgacagaagaagcaccggctaactccgtgccagcagccgcggtaatacggagggtgcgagcgttaatcggaattactgggcgtaaagcgtacgcaggcggtttgttaagcgagatgtgaaagccccgggctcaacctgggaactgcatttcgaactggcaaactagagtgtgatagagggtggtagaatttcaggtgtagcggtgaaatgcgtagagatctgaaggaataccgatggcgaaggcagccacctgggtcaacactgacgctcatgtacgaaagcgtggggagcaaacaggattagataccctggtagtccacgccgtaaacgatgtctactagaagctcggaacttcggttctgtttttcaaagctaacgcattaagtagaccgcctggggagtacggccgcaaggttaaaactcaaatgaattgacgggggcccgcacaagcggtggagcatgtggtttaattcgatgcaacgcgaagaaccttacctacacttgacatacagagaacttaccagagatggtttggtgccttcgggaactctgatacaggt